(19) United States
(12) Patent Application Publication
(10) Pub. No.: US 2020/0306225 A1
(43) Pub. Date: Oct. 1, 2020

(54) STABLE N-((1R,2R)-1-(2,3-DIHYDROBENZO[B][1,4]DIOXIN-6-YL)-1-HYDROXY-3-(PYRROLIDIN-1-YL)PROPAN-2-YL) OCTANAMIDE (2R,3R)-2,3-DIHYDROXYSUCCINATE PREMIX AND PROCESS FOR PREPARATION THEREOF

(71) Applicant: MSN LABORATORIES PRIVATE LIMITED, R&D CENTER, Hyderabad (IN)

(72) Inventors: Thirumalai Rajan SRINIVASAN, Hyderabad (IN); Eswaraiah SAJJA, Hyderabad (IN); Venkat Redby GHOJALA, Hyderabad (IN); Rajeshwar Reddy SAGYAM, Hyderabad (IN); Venkat Reddy MALLEPALLY, Hyderabad (IN); Ravinder reddy KOPPERA, Hyderabad (IN); Adilakshmi SINGAVARAPU, Hyderabad (IN); Srinivasulu RANGINENI, Hyderabad (IN)

(21) Appl. No.: 16/759,710

(22) PCT Filed: Oct. 26, 2018
(86) PCT No.: PCT/IN2018/050693

§ 371 Date: Apr. 27, 2020

(30) Foreign Application Priority Data
Oct. 27, 2017 (IN) IN 201741038102
Feb. 23, 2018 (IN) IN 201841006941

(51) Int. Cl. A61K 31/4025; A61K 47/32; A61K 47/36; A61K 47/02

(52) U.S. Cl. CPC A61K 31/4025; A61K 47/02; A61K 47/36; A61K 47/32

(57) ABSTRACT

The present invention related to stable N-((1R,2R)-1-(2,3-dihydrobenzo[b][1,4] dioxin-6-yl) -1-hydroxy-3-(pyrrolidin-1-yl)propan-2-yl) octanamide (2R,3R)-2,3-dihydroxysuccinate premix of formula (Ia) and its process for preparation thereof. The present invention also related to process for the preparation of N-((1R,2R)-1-(2,3-dihydrobenzo[b][1,4] dioxin-6-yl)-1 -hydroxy-3-(pyrrolidin-1-yl) propan-2-yl) octanamide of formula (I) and pharmaceutically acceptable salts

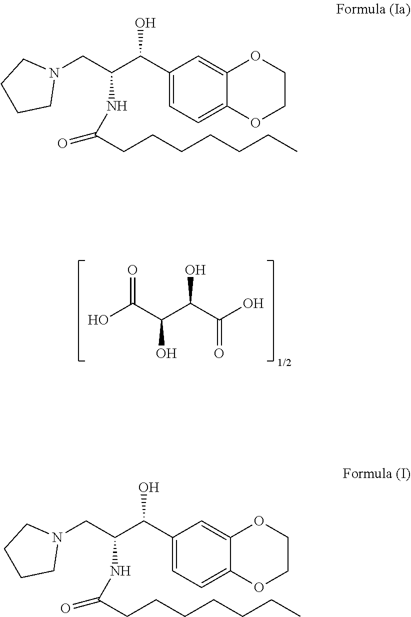

### RELATED APPLICATIONS

This patent application claims the benefit of priority of Indian patent application numbers 201741038102 filed on 27 Oct. 2017 and 201841006941 filed on 23 Feb. 2018 which are incorporated herein by reference.

### FIELD OF THE INVENTION

The present invention relates to a stable N-((1R,2R)-1-(2,3-dihydrobenzo[b][1,4]dioxin-6-yl)-1-hydroxy-3-(pyrrolidin-1-yl)propan-2-yl) octanamide (2R,3R)-2,3-dihydroxysuccinate premix of formula (Ia) and process for the preparation thereof. The chemical structure of said compound is shown below:

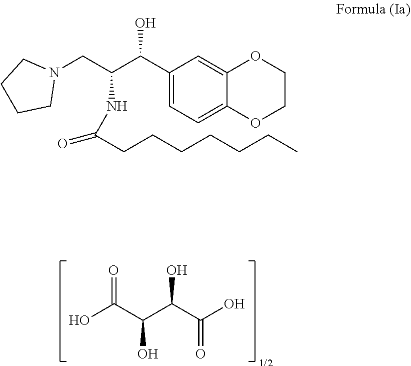

The present invention also related to a process for the preparation of N-((1R,2R)-1-(2,3-dihydrobenzo[b][1,4]dioxin-6-yl)-1-hydroxy-3-(pyrrolidin-1-yl)propan-2-yl) octanamide of formula (I) and its pharmaceutically acceptable salts.

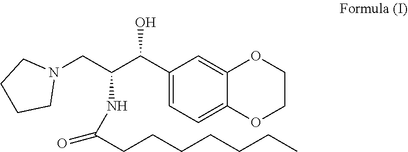 [SMILES: CCCCCCCC(=O)N[C@H](CN1CCCC1)[C@H](O)c1ccc2c(c1)OCCO2]

### BACKGROUND OF THE INVENTION

N-((1R,2R)-1-(2,3-Dihydrobenzo[b][1,4]dioxin-6-yl)-1-hydroxy-3-(pyrrolidin-1-yl) propan-2-yl)octanamide (2R,3R)-2,3-dihydroxysuccinate of formula (Ia) is also known as Eliglustat tartrate (Eliglustat hemitartrate). Eliglustat tartrate is an inhibitor of glucosylceramide synthase, and act as a substrate reduction therapy for gaucher disease type 1 (GD1). Eliglustat tartrate is approved under the brand name of Cerdelga® by USFDA on Aug. 19, 2014 to Genzyme Corporation for oral administration contains 84 mg of Eliglustat, equivalent to 100 mg of Eliglustat hemitartrate in a capsule.

U.S. Pat. No. 7,196,205 B2 discloses a process for the preparation of N-((1R,2R)-1-(2,3-dihydrobenzo[b][1,4]dioxin-6-yl)-1-hydroxy-3-(pyrrolidin-1-yl)propan-2-yl)octanamide comprising: reacting (5S)-5-phenylmorpholin-2-one with 1,4-benzodioxan-6-carboxaldehyde in toluene in presence of molecular sieves for 72 hrs at reflux temperature followed by multiple purification methods like flash chromatography, trituration from ether to produce oxazine adduct (1R,3S,5S,8aS)-1,3-bis-(2′,3′-dihydro-benzo [1,4]dioxin-6′-yl)-5-phenyl -tetrahydro-oxazolo[4,3-c][1,4]oxazin-8-one which is reacted with pyrrolidone and then hydrolyzed followed by purification from flash chromatography to produce an acyclic compound (2S,3R)-3-(2,3-dihydrobenzo[b][1,4]dioxin-6-yl)-3-hydroxy-2-((2-hydroxy-1-phenylethyl)amino)-1-(pyrrolidin-1-yl)propan-1-one. The obtained acyclic compound is reduced and purified from flash chromatography to provide an amine compound (1R,2R)-1-(2,3-dihydrobenzo[b][1,4]dioxin-6-yl)-2-((2-hydroxy-1-phenylethyl)amino)-3-(pyrrolidin-1-yl)propan-1-ol which is debenzylated to form a primary amine compound (1R,2R)-2-amino-1-(2,3-dihydrobenzo [b][1,4]dioxin-6-yl)-3-(pyrrolidin-1-yl) propan-1-ol which is further acylated with N-hydroxy succinimidyl ester to provide Eliglustat of formula (I).

The above reported process suffered from disadvantages such as they consume lot of time, needs expensive chromatography purification methods in each stage and yield of final product is less. Hence, this process is not suitable for industrial scale preparations.

WO 2011066352 A1 discloses crystalline Eliglustat tartrate and process for its preparation thereof.

WO 2016166170 A1 discloses crystalline Eliglustat hydrochloride and its pharmaceutical composition comprising at least one pharmaceutically acceptable excipient.

WO 2016001885 A2 discloses amorphous form of Eliglustat tartrate and its solid dispersion as well as its process for the preparation thereof.

None of the above literature describes stable Eliglustat tartrate premix using Eliglustat free base, tartaric acid and two or more pharmaceutically acceptable excipients without preparation or isolation of crystalline or amorphous form of Eliglustat tartrate of formula (Ia).

Preparation of pharmaceutical dosage forms is often procedurally complex, particularly when combining the final active ingredient with excipients. For example, workability or stability issues may arise when different components of the pharmaceutical dosage form come into intimate contact with one another. It may, thus, be advantageous to supply the manufacturer of pharmaceutical dosage forms with a pre-combined mixture (pre-mix) of excipients and active pharmaceutical ingredient (API) to facilitate and simplify the final processing of dosage forms.

Inventors of the present invention unexpectedly found stable N-((1R,2R)-1-(2,3-dihydrobenzo[b][1,4]dioxin-6-yl)-1-hydroxy-3-(pyrrolidin-1-yl)propan-2-yl) octanamide (2R,3R)-2,3-dihydroxysuccinate premix (also called as “Eliglustat tartrate premix”) with two or more pharmaceutically acceptable excipients.

The premix of the present invention is simple, cost effective and viable for industrial scale preparation methods and readily suitable for formulation or pharmaceutical composition.

There is a significant need in the art to develop an alternate process for the preparation of Eliglustat which is simple, cost effective and suitable for industrial scale up. Further, it is also need in the art to develop stable Eliglustat tartrate premix which improves the performance characteristics of a pharmaceutical product.

### ADVANTAGES OF THE PRESENT INVENTION

Eliglustat tartrate premix prepared by the present invention is quite stable and easy to handle during formulation processing.
Preparation of stable Eliglustat tartrate premix involves the direct use of Eliglustat free base and tartaric acid which avoids the solid isolation of Eliglustat tartrate.
Stabilizing a product with copovidone, magnesium aluminometa silicate and lactose monohydrate
Simple, cost effective and eco-friendly process for the preparation of Eliglustat with high yield.

### BRIEF DESCRIPTION OF THE INVENTION

In a first embodiment, the present invention provides a stable Eliglustat tartrate premix of formula (Ia).

In a second embodiment, the present invention provides a process for the preparation of a stable Eliglustat tartrate premix of formula (Ia).

In a third embodiment, the present invention provides a process for the preparation of Eliglustat and its pharmaceutically acceptable salts.

In a fourth embodiment, the present invention provides the compound of general formula (XII) and its process for the preparation thereof.

In a fifth embodiment, the present invention provides a process for the preparation of the compound of formula (XIIb).

In a sixth embodiment, the present invention provides a process for the preparation of the compound of general formula (XVII).

### BRIEF DESCRIPTION OF THE FIGURES

FIG. 1 Illustrates the X-Ray Powder Diffraction (PXRD) pattern of stable Eliglustat tartrate premix.

FIG.2 Illustrates the Differential Scanning calorimetry (DSC) of Eliglustat tartrate premix.

FIG. 3 Illustrates the X-Ray Powder Diffraction (PXRD) pattern of stable Eliglustat tartrate premix obtained according to example-2.

FIG. 4 Illustrates the X-Ray Powder Diffraction (PXRD) pattern of solid dispersion of Eliglustat tartrate with hydroxy propyl methyl cellulose-acetyl succinate (HPMC-AS)

FIG. 5 Illustrates the powdered X-Ray Diffraction (PXRD) pattern of solid dispersion of Eliglustat tartrate with hydroxy propyl methyl cellulose-E5 (HPMC-E5)

### DETAILED DESCRIPTION OF THE INVENTION

Unless otherwise specified, as used herein the term “suitable solvent” used in the present invention refers to “hydrocarbon solvents” such as n-hexane, n-heptane, cyclohexane, petroleum ether, benzene, toluene, xylene and the like; “ether solvents” such as dimethyl ether, diisopropyl ether, diethyl ether, methyl tert-butyl ether, 1,2-dimethoxy ethane, tetrahydrofuran, 1,4-dioxane and the like; “ester solvents” such as methyl acetate, ethyl acetate, isopropyl acetate, n-butyl acetate and the like; “polar-aprotic solvents such as dimethylacetamide(DMAc), dimethylformamide (DMF), dimethylsulfoxide (DMSO), N-methylpyrrolidone (NMP) and the like; “chloro solvents” such as dichloromethane, dichloroethane, chloroform, carbon tetrachloride and the like; “ketone solvents” such as acetone, methyl ethyl ketone, methyl isobutyl ketone and the like; “nitrile solvents” such as acetonitrile, propionitrile, isobutyronitrile and the like; “alcoholic solvents” such as methanol, ethanol, n-propanol, isopropanol, n-butanol, isobutanol, t-butanol and the like; “polar solvents” such as water or mixtures thereof.

As used herein the term “suitable base” refers to the bases selected from alkali metal hydroxides like sodium hydroxide, potassium hydroxide; alkali metal carbonates like sodium carbonate, potassium carbonate and alkali metal bicarbonates like sodium bicarbonate, potassium bicarbonate and the like; organic bases like methylamine, ethylamine, isopropylamine, diisopropyl ethylamine, triethylamine, 1,8-bis(dimethylamino)naphthalene, alkali metal alkoxides like sodium tertiary butoxide, potassium tertiary butoxide, pyridine, 4-dimethylaminopyridine, ammonia or their aqueous solution; ammonium bases such as ammonium carbonate, ammonium hydrogen carbonate or ammonium bicarbonate, ammonium sulfate, ammonium hydrogen sulfate and the like.

As used herein the term “suitable acid” refers to the acid selected from inorganic acids like hydrochloride, hydrobromide, hydroiodide, sulfuric acid; organic acids like acetic acid, methanesulfonic acid, p-toluenesulfonic acid, trifluoroacetic acid.

As used herein, the term suitable “coupling agent” is selected from but not limited to 1, 1′-carbonyl diimidazole (CDI), N,N′-dicyclohexylcarbodiimide (DCC), N,N′-diisopropyl carbodiimide (DIC), 1-ethyl-3-(3-dimethylaminopropyl)carbodiimide hydrochloride (EDC.HCl), 1-[bis(dimethylamino)methylene]-1H-1,2,3-triazolo[4,5 -b]pyridinium 3-oxid hexafluoro phosphate (HATU), 2-(1H-benzotriazol-1-yl)-1,1,3,3-tetramethyluronium hexafluoro phosphate (HBTU), 1H-benzo triazolium 1[bis(dimethylamino)methylene]-5chloro-hexafluorophosphate (1-) 3-oxide (HCTU), O-(benzotriazol-1-yl)-N,N,N′,N′-tetramethyl uronium tetrafluoroborate (TBTU), alkyl/aryl haloformates selected from but not limited to ethyl chloroformate, benzylchloroformate; diphenylphosphoroazidate (DPPA), thionyl chloride, oxalyl chloride, phosphorous oxychloride, phosphorous pentachloride, 4-methyl-2-oxopentanoyl chloride (i-BuCOCOCl), (benzotriazol-1-yloxy) tris(dimethylamino) phosphonium hexafluorophosphate (BOP), benzotriazol-1-yl-oxy tripyrrolidinophosphonium hexafluorophosphate (PyBOP), methane sulfonyl chloride, p-toluenesulfonyl chloride and the like.

As used herein, the term “suitable protecting agent” used in the present invention refers to a group of reagents independently selected such that they are capable of protecting the hydroxy groups of various compounds of the present invention with suitable O-protecting groups selected from but not limited to silyl protecting groups, 3,4-dihydro-2H-pyranyl (DHP), tetrahydropyranyl (THP), tetrahydrofuranyl (THF), methyl, acetyl, benzyl, benzoyl, benzyloxycarbonyl (Cbz), trifluoroacetyl, pivaloyl, allyl, methoxymethyl (MOM), ethoxyethy (EE), methoxyethoxymethyl (MEM), p-methoxybenzyl (PMB), methylthiomethyl (MTM), triphenylmethyl (trityl), methoxytrityl (MMT), dimethoxytrityl (DMT), benzyloxymethyl (BOM), tert.butoxy carbonyl (Boc) and the like; the preferable silyl protecting groups can be selected from but not limited to trialkylsilyl, triarylsilyl, alkyl/aryl silyl such as trimethylsilyl (TMS), triethylsilyl (TES), triisopropylsilyl (TIPS), tert.butyldimethylsilyl (TBS or TBDMS), tri-iso-propylsilyloxymethyl (TOM), tert-butyldiphenylsilyl (TBDPS) and the like.

The suitable protecting agent is selected from but not limited to trialkyl silyl halides such as trimethylsilyl chloride (TMSCl), triethylsilyl chloride (TESCl), triisopropylsilyl chloride (TIPSCl), tert-butyldimethylsilyl chloride (TBDMSCl), tert-butyldiphenylsilyl chloride (TBDPSCl) and the like; trialkyl silyl triflates such as trimethylsilyl triflate (TMSOTf), triethylsilyl triflate (TESOTf), triisopropylsilyl triflate (TIPSOTf), tert-butyldimethyl silyl triflate (TBDMSOTf or TBSOTf), tert-butyldiphenylsilyl triflate (TBDPSCl) and the like; N,0-bis(trimethylsilyl)acetamide (BSA), hexemethyldisilazane (HMDS), dihydropyran, 2-chloro tetrahydrofuran, diazomethane, methyl halides, acetyl chloride, acetic anhydride, benzyl halides, benzoyl chloride, benzoic anhydride, benzyloxycarbonyl chloride, trifluoroacetyl chloride, trifluoroacetic anhydride, tert-butyl acetyl chloride, tert-butyl acetic anhydride, allyl halides, methoxymethyl halides, ethoxyethyl halides, methoxyethoxymethyl halides, p-methoxybenzyl halides, methylthiomethyl halides, trityl halides, benzyloxymethyl halides, di-tert.butyl dicarbonate and the like.

The “suitable deprotecting agent” can be selected based on the protecting group employed. The suitable deprotecting agent can be selected from but not limited to acids such as hydrochloric acid, hydrobromic acid, phosphoric acid, acetic acid, formic acid, trifluoroacetic acid, methane sulfonic acid, p-toluene sulfonic acid, camphor sulfonic acid and the like, bases such as alkali metal hydroxides, alkali metal carbonates, cesium carbonate/imidazole, alkali metal bicarbonates, ammonia, cerium ammonium nitrate (CAN), 1,8-diazabicyclo[5.4.0]undec-7-ene (DBU), hydrogenating agents such as Pd, Pd/C, Pd(OH)₂/C (Pearlman's catalyst), palladium acetate, platinum oxide (PtO₂), platinum black, sodium borohydride, BF₃-etherate, Raney-Ni, triethylsilane, trimethylsilyl halides, copper(II) chloride dihydrate and the like; fluoride ion sources such as sodium fluoride (NaF), potassium fluoride (KF), tetra butyl ammonium fluoride (TBAF), HF-pyridine, HF-triethyl amine, ammonium fluoride; trifluoromethane sulfonic acid (triflic acid), tris(dimethylamino)sulfonium difluorotrimethylsilicate (TASF), 2,3-dichloro-5,6-dicyano-1,4-benzoquinone (DDQ), acetyl chloride/methanol, N-iodosuccinimide in methanol; Lewis acids such as AlCl₃, AlBr₃, BBr₃, ZnBr₂, TiCl₄ and the like.

The term “Azide source” is selected from but not limited to lithium azide (LiN₃) or sodium azide (NaN₃).

As used herein, the term “reducing agent” is selected from but not limited to sodium borohydride, lithium aluminum hydride, Pd/C, sodium triacetoxyborohydride (NaBH(OAc)₃).

As used herein, the term “premix” refers to a solid containing mixture of Eliglustat, pharmaceutically acceptable salt with two or more pharmaceutical excipients.

As used herein, the term “pharmaceutically acceptable salt” refer to the salts prepared from pharmaceutically acceptable inorganic or organic acids selected from HCl, HBr, HI, methanesulfonic acid, p-toluenesulfonic acid, oxalic acid, tartaric acid, succinic acid and the like.

The term “stable” herein means Eliglustat tartrate premix that substantially does not convert to any other polymorphic form.

In first embodiment, the present invention provides a stable Eliglustat tartrate premix comprising Eliglustat tartrate and two or more pharmaceutical acceptable excipients.

In an aspect of first embodiment, the present invention provides a stable Eliglustat tartrate premix, comprising: at least two or more pharmaceutical acceptable excipients.

In an aspect of first embodiment, the present invention provides a stable Eliglustat tartrate premix which is prepared by combining Eliglustat, tartaric acid and two or more pharmaceutical acceptable excipients. wherein, the pharmaceutical acceptable excipients include (but are not limited to) binders, diluents, disintegrants, surfactants and lubricants. Suitable binders that can be include (but are not limited to) polyvinylpyrolidone, copovidone, starches such as pregelatinized starch, cellulose derivatives such as hydroxypropylmethyl cellulose, ethylcellulose, hydroxypropylcellulose and carboxymethylcellulose and their salts, gelatine, acacia, agar, alginic acid, carbomer, chitosan, dextrates, cyclodextrin, dextrin, glycerol dibehenate, guargum, hypromellose, maltodextrin, poloxamer, polycarbophil, polydextrose, polyethylene oxide, polymethacrylates, sodium alginate, sucrose, mixtures thereof and the like; suitable diluents that can be include (but are not limited to) anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol starch, calcium lactate or mixtures of diluents; suitable disintegrants that can be include (but are not limited to) magnesium aluminometa silicate (or magnesium aluminum silicate), starch, pregelatinized starch, sodium starch glycolate, crospovidone, croscarmellose sodium, low-substituted hydroxypropyl cellulose, alginic acid, carboxy methyl cellulose sodium, sodium alginate, calcium alginate and chitosan; suitable lubricants that can be include (but are not limited to) magnesium stearate, stearic acid, palmitic acid, talc, and aerosil. Suitable surfactants that can be include (but are not limited to) polysorbate 80, polyoxyethylene sorbitan, polyoxyethylene-polyoxy-propylene copolymer and sodium lauryl sulphate.

In one aspect of first embodiment, the present invention provides a stable Eliglustat tartrate premix with two or more pharmaceutical acceptable excipients selected from copovidone, lactose monohydrate, and magnesium aluminometa silicate.

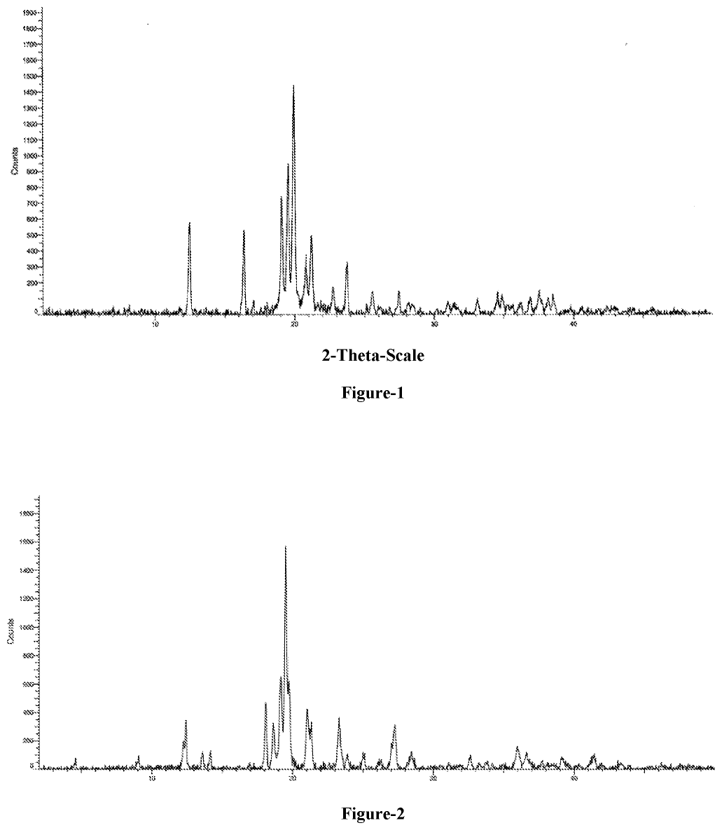

In second aspect of first embodiment, the present invention provides stable Eliglustat tartrate premix characterized by powdered X-Ray Diffraction Pattern as illustrated in FIG. 1.

In third aspect of first embodiment, the present invention provides stable Eliglustat tartrate premix characterized by Differential Scanning calorimetry which exhibits the glass transition between about 45° C. to about 55° C. and the same is illustrated in FIG. 2.

In fourth aspect of first embodiment, the present invention provides stable Eliglustat tartrate premix having the bulk density between about 0.1 gm/ml to about 0.9 gm/ml. Prefarebly, between about 0.5 gm/ml to about 0.7 gm/ml.

In fifth aspect of first embodiment, the present invention provides stable Eliglustat tartrate premix contains less than about 0.15% of one or more impurities selected from pyrrolidine amine impurity, hexanamide impurity, N-oxide impurity, nonamide impurity, amide impurity, decanamide impurity and dioctanoyl impurity. Prefarebly, less than about 0.1%. Further, prefarebly, less than about 0.05%. More prefarebly, substantially free from one or more impurities selected from pyrrolidine amine impurity, nonamide impurity, amide impurity, decanamide impurity and dioctanoyl impurity.

The chemical structures of said impurities are as follows:

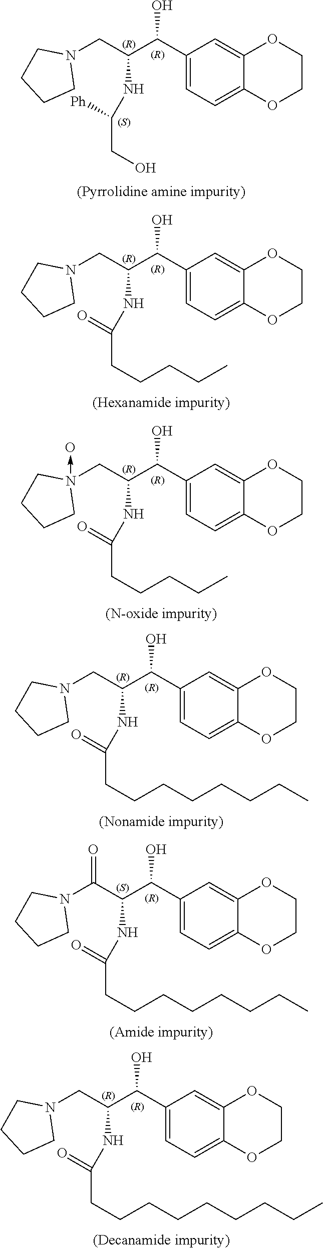

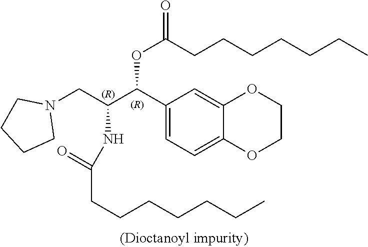

The term “substantially free” refers to stable Eliglustat tartrate premix contains less than about 0.01% of any of impurities.

In sixth aspect of first embodiment, the present invention provides stable Eliglustat tartrate premix with co-povidone, lactose monohydrate and magnesium aluminometa silicate. Wherein the starting material Eliglustat free base is a crystalline or amorphous form and the total weight ratio of excipients may vary from 10-70%. Preferably about 55% of excipients on the total weight of Eliglustat tartrate premix.

Wherein the said 55% of excipients contains about 26.5% of lactose monohydrate, about 19.0% of magnesium aluminometa silicate and about 9.5% of copovidone.

In seventh aspect of first embodiment, the present invention provides Eliglustat tartrate premix of formula (Ia) is stable under stress conditions at 60° C. for 24 hours, 10 tons of pressure and under UV light at 254 nm for 24 hours.

In eighth aspect of first embodiment, the present invention provides the direct use of crystalline or amorphous form of Eliglustat free base and tartaric acid in the preparation of stable Eliglustat tartrate premix.

In a second embodiment, the present invention provides a process for the preparation of a stable Eliglustat tartrate premix, comprising:
a) dissolving Eliglustat free base in a suitable solvent,
b) adding tartaric acid to the solution obtained in step-a)
c) optionally filtering the solution,
d) adding two or more pharmaceutical acceptable excipients to the solution obtained in step-a) or step-b),
e) isolating stable Eliglustat tartrate premix.
   wherein, in step-a) the suitable solvent is selected from “alcoholic solvents” such as methanol, ethanol, isopropanol, n-propanol, n-butanol, iso-butanol, ethylene glycol and the like; “ester solvents” such as ethyl acetate, methyl acetate, n-butyl acetate, isobutyl acetate, sec-butyl acetate, isopropyl acetate and the like, “ether solvents” such as tetrahydrofuran, diethylether, methyl tert-butyl ether, 1,4-dioxane and the like; “hydrocarbon solvents” such as toluene, xylene, cyclohexane, n-hexane, n-heptane, n-pentane and the like; “chloro solvents” such as methylene chloride, ethylene dichloride, carbon tetrachloride, chloroform and the like; “polar aprotic solvents” such as dimethylformamide, dimethylacetamide, dimethylsulfoxide and the like; “nitrile solvents” such as acetonitrile, isobutyronitrile and the like; “ketone solvents” such as acetone, methyl isobutyl ketone, methyl ethyl ketone; polar solvent such as water and mixtures thereof; in step-d) the suitable pharmaceutically acceptable excipients include (but are not limited to) binders, diluents, disintegrants, surfactants and lubricants. Suitable binders that can be include (but are not limited to) polyvinylpyrolidone, copovidone, starches such as pregelatinized starch, cellulose derivatives such as hydroxypropylmethyl cellulose, ethylcellulose, hydroxypropylcellulose and carboxymethylcellulose and their salts, gelatine, acacia, agar, alginic acid, carbomer, chitosan, dextrates, cyclodextrin, dextrin, glycerol dibehenate, guargum, hypromellose, maltodextrin, poloxamer, polycarbophil, polydextrose, polyethylene oxide, polymethacrylates, sodium alginate, sucrose, mixtures thereof and the like; suitable diluents that can be include (but are not limited to) anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol starch, calcium lactate or mixtures of diluents; suitable disintegrants that can be include (but are not limited to) magnesium aluminometa silicate (or magnesium aluminum silicate), starch, pregelatinized starch, sodium starch glycolate, crospovidone, croscarmellose sodium, low-substituted hydroxypropyl cellulose, alginic acid, carboxy methyl cellulose sodium, sodium alginate, calcium alginate and chitosan; suitable lubricants that can be include (but are not limited to) magnesium stearate, stearic acid, palmitic acid, talc, and aerosil; suitable surfactants that can be include (but are not limited to) polysorbate 80, polyoxyethylene sorbitan, polyoxyethylene-polyoxy-propylene copolymer and sodium lauryl sulphate; in step-e) the isolation can be carried out by removing the solvent by the techniques like filtration, distillation, solvent dry distillation, spray drying, agitated thin film drying (“ATFD”), freeze drying (lyophilization).

In one aspect of second embodiment, the present invention provides a process for the preparation of stable Eliglustat tartrate premix of formula (Ia), comprising:
a) dissolving Eliglustat free base in methanol,
b) adding tartartic acid to a solution obtained in step-a)
c) adding co-povidone to a solution obtained in step-b),
d) adding mixture of magnesium aluminometa silicate & lactose monohydrate to a reaction mixture obtained in step-c),
e) removing the solvent to produce stable Eliglustat tartrate premix.

In a third embodiment, the present invention provides a process for the preparation of Eliglustat of formula (I), comprising:
a) reacting the compound of formula (VII) with chlorinating agent in a suitable solvent and followed by reacting the resulting compound with pyrrolidine to provide a compound of formula (VIII)

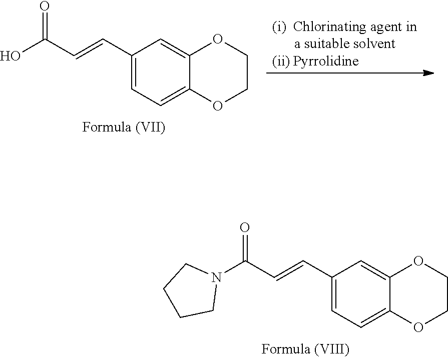 [SMILES: O=C(O)/C=C/c1ccc2c(c1)OCCO2 ; O=C(/C=C/c1ccc2c(c1)OCCO2)N1CCCC1]

b) converting the compound of formula (VIII) to provide the compound of general formula (IX)

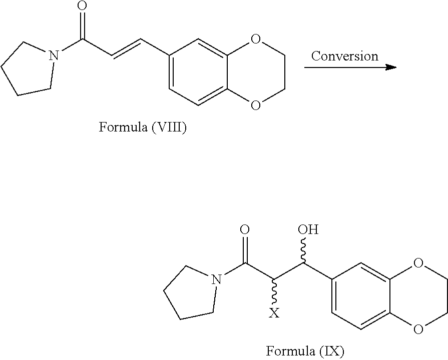

wherein X refers to halogen selected from chlorine, bromine or iodine.
c) optionally protecting the hydroxy group of compound of general formula (IX) with a suitable protecting agent to provide the compound of general formula (IXp)

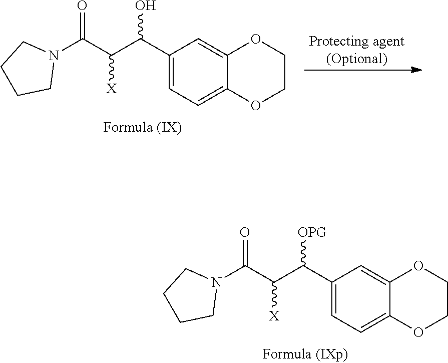

wherein, PG refers to protecting group.
d) reacting the compound of general formula (IX) or the compound of general formula (IXp) with suitable azide source to provide compound of general formula (X)

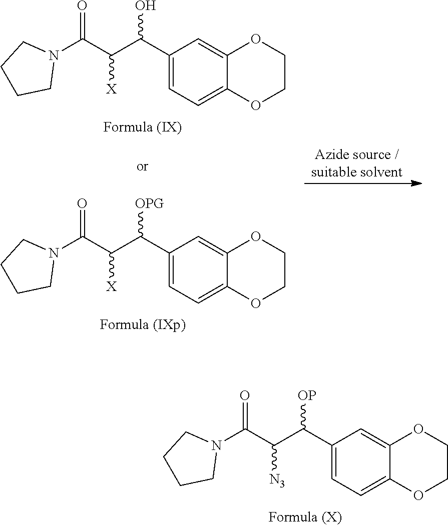

wherein, P refers to hydrogen or a hydroxy protecting group.
e) reacting the compound of general formula (X) with suitable reducing agent in a suitable solvent to provide the compound of formula (XI)

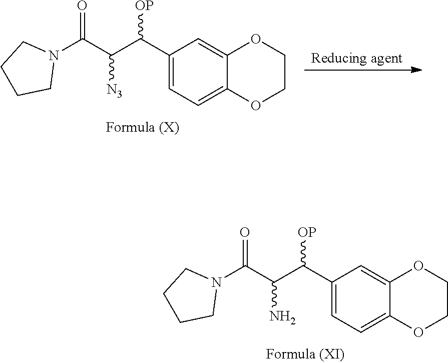

f) treating the compound of general formula (XI) with a suitable resolving agent in a suitable solvent to provide a diastereomeric salt of general formula (XII)

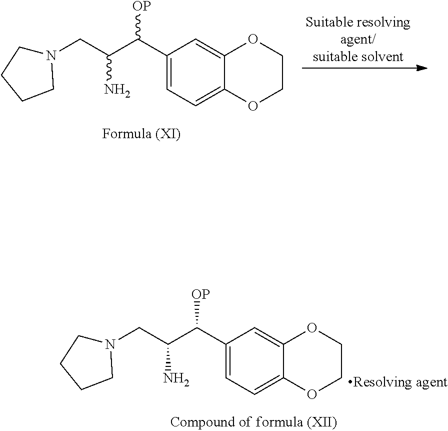

g) treating a diastereomeric salt of general formula (XII) with a base in a solvent to provide the compound of formula (XIIa)

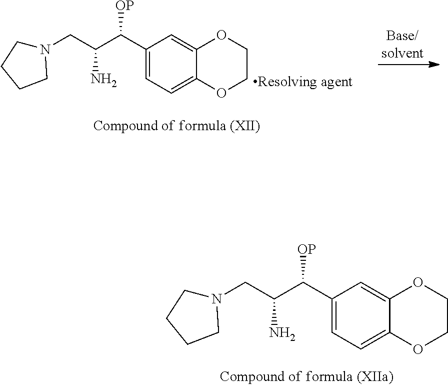

h) reacting a compound of formula (XIIa) with octanoic acid derivative to provide compound of formula (Ib)

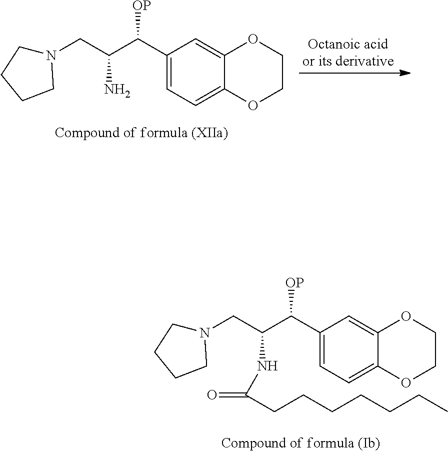

i) optionally, deprotecting the compound of formula (Ib) with suitable acid or base to provide Eliglustat of formula (I),
j) optionally, converting the product obtained in step-h) or step-i) to provide an acid addition salt of Eliglustat.
   wherein in step-a), the suitable chlorinating agent is selected from thionyl chloride or oxalyl chloride, PCl₃, PCl₅ and CCl₄; the suitable solvent is selected from hydrocarbon solvents, polar aprotic solvents, ester solvents, ether solvents, nitrile solvents, alcohol solvents, chloro solvents or mixtures. The obtained acyl chloride is then reacted with pyrrolidine in presence of water or aqueous or non aqueous organic solvent to get the compound of formula (VIII); in step-b), the conversion is carried out by the reaction of the compound of formula (VIII) with a reagent selected from sodium periodate (NaIO₄), osmium tetroxide and the like followed by treating with lithium chloride, lithium bromide or lithium iodide in a suitable solvent; in step-c) the suitable protecting agent is same as defined in hereinbefore; in step-d) the suitable azide source is selected from sodium azide, lithium azide or potassium azide and the like; the suitable solvent is selected from hydrocarbon solvents, nitrile solvents, ester solvents, ether solvents, polar aprotic solvents or mixture thereof; in step-e) the suitable reducing agent is selected from sodium borohydride, lithium aluminum hydride, trialkyl silylhydride such as trimethylsilyl hydride, triethylsilyl hydride and the like; in step-f) the suitable resolving agent is selected from substituted or unsubstituted N-acetyl-L-leucine, N-acetyl-L-tyrosine; D- or L-tartartic acid, di-para-tolyl D-tartratic acid, di-benzoyl D-tartaric acid, (+)-camphor-10-sulfonic acid and (+)-α-phenyl ethylamine, mandelic acid, malic acid and the like; suitable solvent is selected from hydrocarbon solvents, chloro solvents, ester solvents, ether solvents, alcohol solvents, nitrile solvents or mixture thereof; in step-g) the suitable base is selected from alkali metal hydroxides like sodium hydroxide, potassium hydroxide; alkali metal carbonates like sodium carbonate, potassium carbonate and alkali metal bicarbonates like sodium bicarbonate, potassium bicarbonate and the like; alkali metal alkoxides like sodium tert-butoxide, potassium tert-butoxide; suitable solvent is same as defined hereinbefore; in step-h), the octanoic acid derivative is selected from octanoic acid halide such as octanoic acid chloride or octanoic acid bromide, octanoic acid ester derivatives such as 2,5-dioxopyrrolidin-1-yl octanoate, pentafluorophenyl octanoate and the like; in step-i) the suitable acid is selected from organic or inorganic acids; the suitable base is selected from alkali metal hydroxides; alkali metal carbonates and alkali metal bicarbonates; alkali metal alkoxides; in step-j) the acid addition salt is selected from organic or inorganic acids; prefarebly organic acids; more prefarebly tartaric acid (also called as (2R,3R)-2,3-dihydroxysuccinic acid).

In an aspect of third embodiment, the present invention provides a process for the preparation of Eliglustat of formula (I) or its salts, comprising: reacting the compound of formula (XIIb) with perfluorophenyl octanoate in presence of a base in a suitable solvent to provide the compound of formula (I) or its salts.

The process shown below:

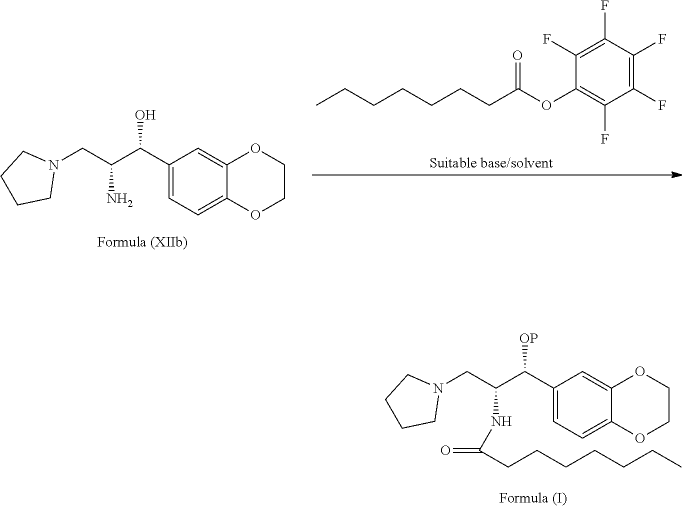

wherein, the suitable base and suitable solvent are same as defined hereinbefore.

In one aspect of third embodiment, the present invention provides a process for the preparation of Eliglustat of formula (I), comprising:
a) reacting (E)-3-(2,3-dihydrobenzo[b][1,4]dioxin-6-yl)acrylic acid of formula (VII) with thionyl chloride in toluene and followed by reacting the resulting compound with pyrrolidine to provide (E)-3-(2,3-dihydrobenzo[b][1,4]dioxin-6-yl)-1-(pyrrolidin-1-yl)prop-2-en-1-one of formula (VIII);
b) converting (E)-3-(2,3-dihydrobenzo [b][1,4]dioxin-6-yl)-1-(pyrrolidin-1-yl)prop-2-en -1-one of formula (VIII) to 2-bromo-3-(2,3-dihydrobenzo [b][1,4]dioxin-6-yl)-3-hydroxy-1-(pyrrolidin-1-yl)propan-1-one of formula (IXa);
c) reducing the 2-bromo-3-(2,3-dihydrobenzo[b][1,4]dioxin-6-yl)-3-hydroxy-1-(pyrrolidin-1-yl)propan-1-one of formula (IXa) with lithium aluminum hydride to provide 2-amino-1-(2,3-dihydrobenzo [b][1,4]dioxin-6-yl)-3-(pyrrolidin-1-yl)propan -1-olof formula (XIa);
d) resolving a compound of formula (XIa) with N-acetyl-L-leucine in toluene to provide diastereomeric salt of formula (XIIa);

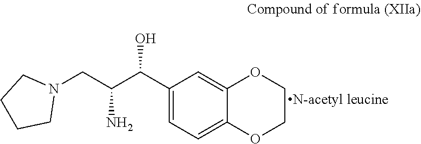 [SMILES: N[C@H](CN1CCCC1)[C@H](O)c1ccc2c(c1)OCCO2]

e) treating diastereomeric salt of formula (XIIa) with sodium hydroxide in methylene chloride and water to provide the compound of formula (XIIb);
f) reacting the compound of formula (XIIb) with perfluorophenyl octanoate in presence of a base in a suitable solvent to provide the Eliglustat of formula (I).

In second aspect of third embodiment, the present invention provides alternate routes for the preparation of compound of formula (VII) as schematically as shown below:

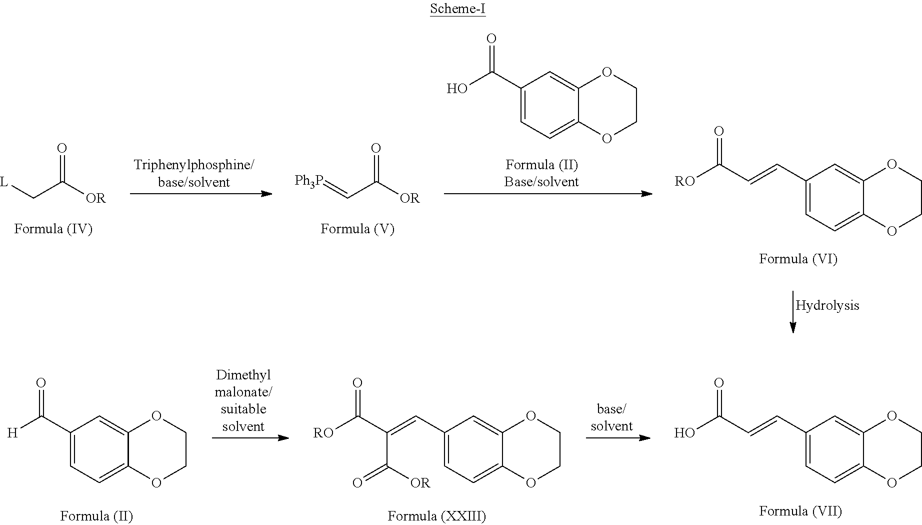

wherein, L is a leaving group such as chlorine, bromine, iodine or fluorine or alkyl or aryl sulfonyloxy groups. R is an alkyl group having C₁-C₅ carbon atoms.

In third aspect of third embodiment, the present invention provides diastereomeric salt of general formula (XII)

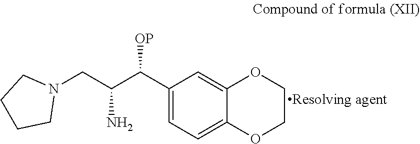

wherein, P is hydrogen or protecting group; resolving agent is selected from N-acetyl-L-leucine, N-acetyl-L-tyrosine, D- or L-tartartic acid, di-para-tolyl D-tartratic acid, di-benzoyl D-tartaric acid, (+)-camphor-10-sulfonic acid and (+)-α-phenyl ethylamine, mandelic acid, malic acid and the like.

In fourth aspect of third embodiment, the present invention provides prefarebly solid form of diastereomeric salt of (1R,2R)-2-amino-1-(2,3-dihydrobenzo)[b][1,4]dioxin-6-yl)-3-(pyrrolidin-1-yl)propan-1-ol with N-acetyl leucine of formula (XIIa).

In fifth aspect of third embodiment, the present invention provides a process for the preparation of diastereomeric salt of general formula (XII), comprising: treating the compound of general formula (XI) with a suitable resolving agent in a suitable solvent to provide the diastereomeric salt of general formula (XII)

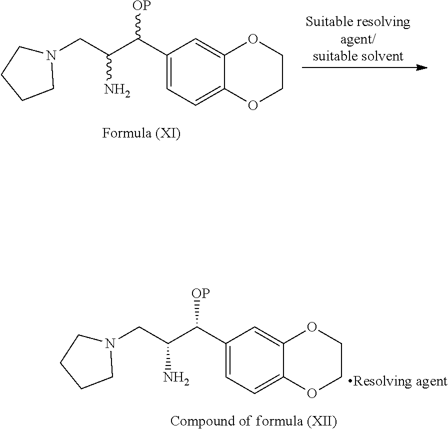

wherein, suitable resolving agent and suitable solvent are same as defined hereinbefore.

In a fourth embodiment, the present invention provides a process for the preparation of the compound of formula (XIIb), comprising:
a) reacting the compound of general formula (XVII) with the compound of general formula (XVIII) in presence of lithium source in a suitable solvent to provide the compound of general formula (XIX)

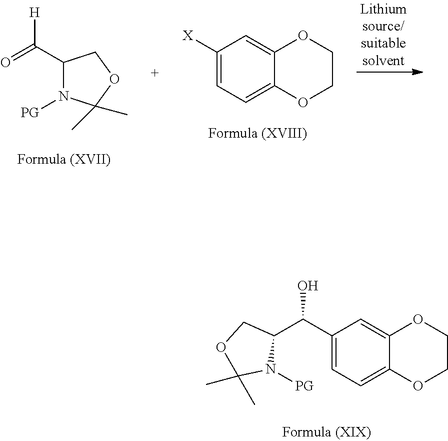

b) treating the compound of general formula (XIX) with suitable acid in a suitable solvent to provide the compound of general formula (XX)

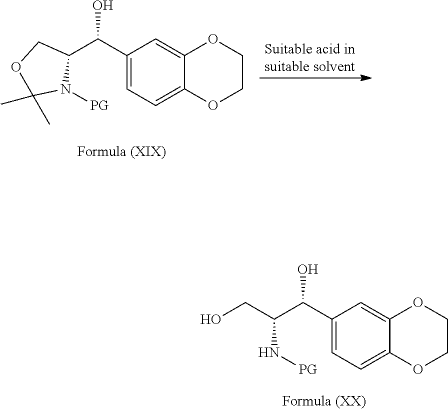

c) reacting the compound of general formula (XX) with suitable acid chloride in presence of suitable base in a suitable solvent to provide the compound of general formula (XXI)

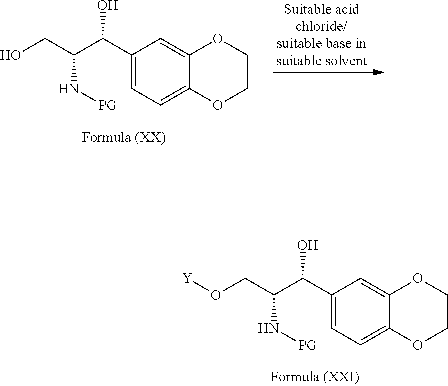

wherein, Y refers to alkyl or aryl sulfonyl group
d) reacting the compound of general formula (XXI) with the pyrrolidine to provide the compound of formula (XXII)

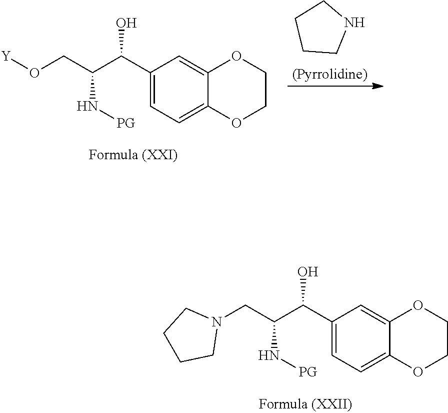

e) treating the compound of general formula (XXII) with the suitable acid in a suitable solvent to provide the compound of formula (XIIb) or its acid addition salts

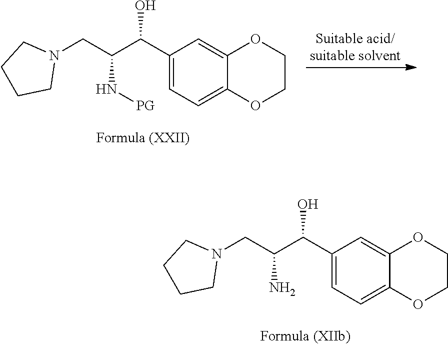

wherein PG refers to suitable protecting group and X refers to halogen atom such as bromine, chlorine, iodine or fluorine.
wherein in step-a), the suitable lithium source is selected from n-butyl lithium, tert-butyl lithium, lithium iodide and the like; the suitable solvent selected from ether solvents, ester solvents, nitrile solvents, polar aprotic solvents, alcohol solvents, chloro solvents or mixtures; in step-b), the suitable acid is selected from p-toluenesulfonic acid, methanesulfonic acid, ethanesulfonic acid, benzenesulfonic acid or mineral acids such as hydrochloride, acetonitrile -hydrochloride, acetone-hydrochloride, ethyl acetate-hydrochloride, hydrobromide, nitric acid, sulfuric acid and the like; the suitable solvent is selected from alcohol solvents, ether solvents, ester solvents, nitrile solvents, polar aprotic solvents or mixture thereof; in step-c), the suitable acid chloride is selected from p-toluenesulfonic acid chloride, methanesulfonic acid chloride, ethanesulfonic acid chloride, benzenesulfonic acid chloride and the like; the suitable base is selected from organic or inorganic bases; the organic base is selected from methylamine, ethylamine, n-propylamine, triethylamine, diisopropylethylamine, n-methylmorpholine, pyridine or mixtures thereof; the inorganic base is selected from alkali metal hydroxides like sodium hydroxide, potassium hydroxide; alkali metal carbonates like sodium carbonate, potassium carbonate and alkali metal bicarbonates like sodium bicarbonate, potassium bicarbonate and the like; alkali metal alkoxides like sodium tert-butoxide, potassium tertbutoxide.ke; the suitable solvent is selected from chloro solvents, ether solvents, ester solvents, alcohol solvents, nitrile solvents or mixture thereof; in step-d), the suitable solvent is selected from water, alcohol solvents, nitrile solvents or mixture thereof; in step-e), the suitable acid is selected from mineral acids such as HCl, aqueous HCl, acetonitrile-HCl, methanol-HCl, ethanol-HCl, isopropanol-HCl, ethereal-HCl, HBr, nitric acid, sulfuric acid or organic acids such as acetic acid, trifluoro acetic acid, methanesulfonic acid, p-toluenesulfonic acid and the like; the suitable solvent is selected from chloro solvents, ether solvents, ester solvents, alcohol solvents, nitrile solvents or mixture thereof.

In a sixth embodiment, the present invention provides a process for the preparation of the compound of formula (XVII), comprising:
a) reacting the compound of formula (XIV) with N,O-dimethylhydroxylamine HCl in presence of suitable coupling agent, suitable base in a suitable solvent to provide the compound of formula (XV);

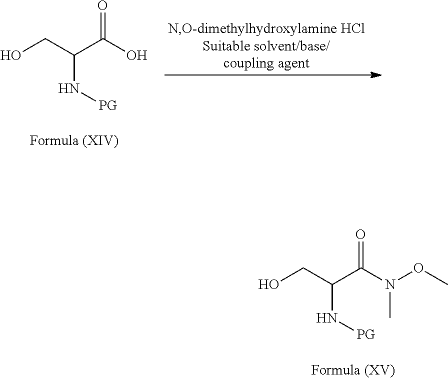

b) reacting the compound of formula (XV) with 2,2-dimethoxypropane in presence of suitable catalyst, suitable base in a suitable solvent to provide the compound of formula (XVI);

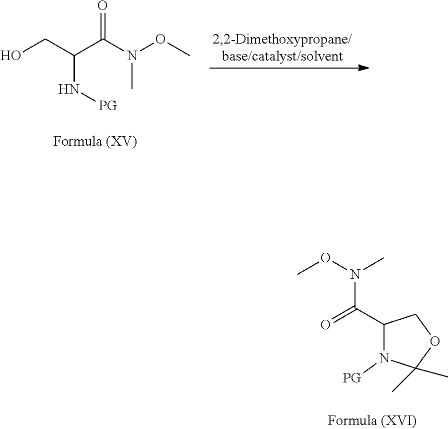

c) reduction of the compound of formula (XVI) with the suitable reducing agent to provide the compound of formula (XVII)

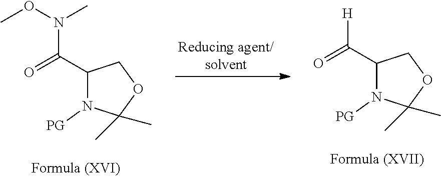

wherein, PG refers to Protecting Group.
wherein in step-a) the suitable base is selected from organic bases such as N-methylmorpholine (NMM), triethylamine (TEA), diisopropylethylamine (DIPEA) and the like; the suitable coupling agent is selected from N,N-carbonyldiimidazole (CDI); alkyl and aryl carbodiimides such as N,N-diisopropylcarbodiimide (DIC), N,N-dicyclohexyl carbodiimide (DCC), 1-ethyl-3-(3-dimethylaminopropyl)carbodiimide hydrochloride (EDC.HCl), ditolyl carbodiimide optionally in combination with hydroxybenzotriazole or N-hydroxysuccinimide (NHS) or N-hydroxysulfosuccinimide (Sulfo-NHS); carbonyl-di-1,2,4-triazole; alkyl and aryl halo formates such as ethyl chloroformate, phenyl chloroformate, benzyl chloroformate; carbonates having the formula R₁—O—CO—O—R₂, wherein “R₁” and “R₂” are independently selected from branched or unbranched C₁-C₄ alkyl or substituted or unsubstituted phenyl group; the suitable base is selected form organic or inorganic bases; and the suitable solvent is selected from polar aprotic solvents, ketone solvents, ester solvents, chloro solvents, ether solvents, hydrocarbon solvents, nitrile solvents, polar solvents or mixtures; in step-b) the suitable catalyst is selected from Lewis acids such as BF₃.etherate, AlCl₃, TiCl₄, BCl₃ and the like; the suitable base is selected form organic or inorganic bases; and the suitable solvent is selected from polar aprotic solvents, ketone solvents, ester solvents, chloro solvents, ether solvents, hydrocarbon solvents, nitrile solvents, polar solvents or mixtures; in step-c) the suitable reducing agent is selected from sodium bis(2-methoxyethoxy)aluminum hydride (Vitride), diborane, borane-dimethyl sulfide, borane-THF complex, sodium tri-acetoxyborohydride, sodium cyanoborohydride, NaBH₄, NaBH₄/BF₃.diethyl ether, LiBH₄, LiAlH₄ and the like; and the suitable solvent is selected from alcoholic solvent, ether solvents, ester solvents, hydrocarbon solvents, polar solvents, polar aprotic solvents or mixtures.
The process of the present invention is schematically as shown as below scheme:

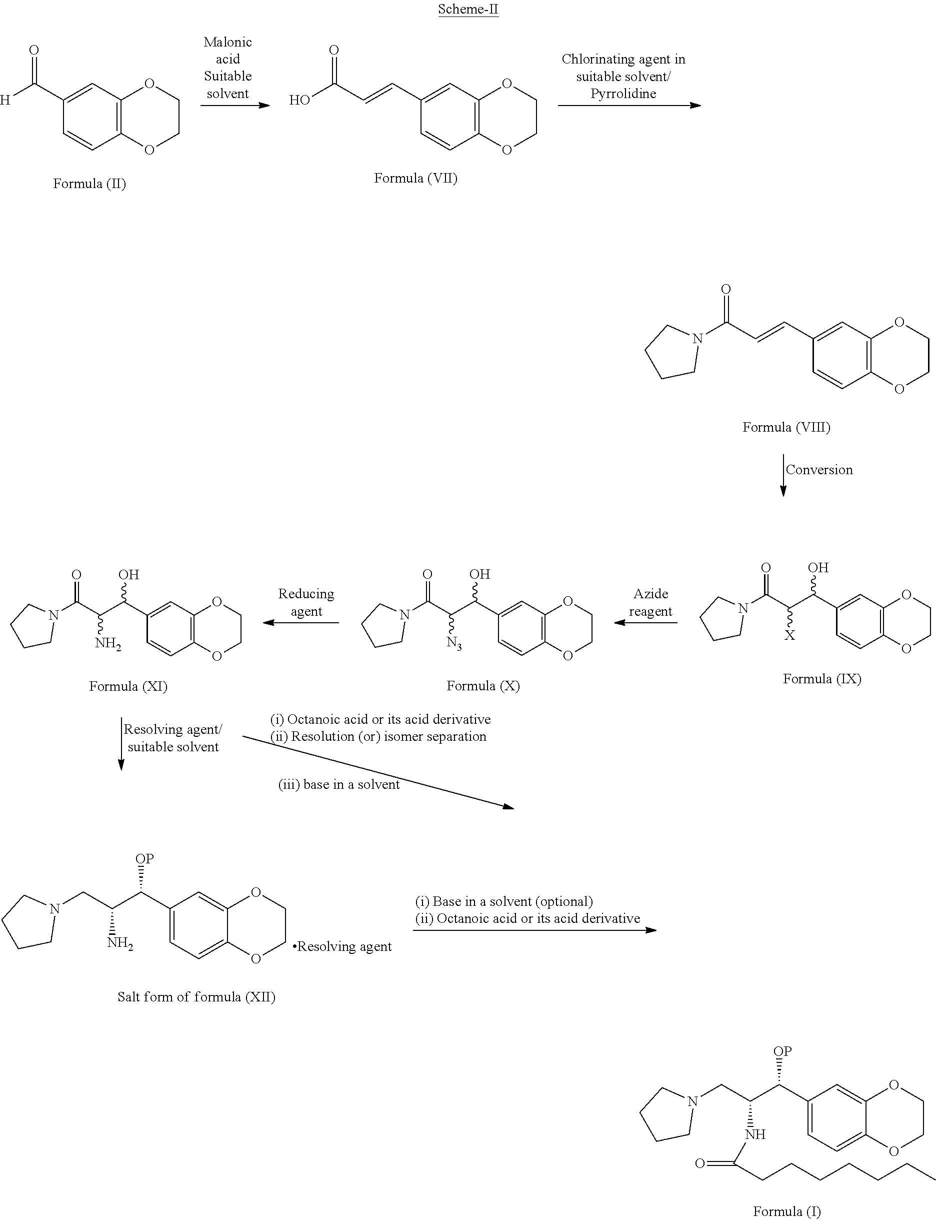

wherein X refers to halogen such chlorine, bromine or iodine.

Differential scanning calorimetric (DSC) analysis was performed with TA DSC Q2000 model calorimeter.

PXRD analysis of Eliglustat tartrate premix of the present invention was carried out using BRUKER-AXS D8 Advance X-Ray diffractometer using Cu-Ka radiation of wave-length 1.5406 A° and at continuous scan speed of 0.03°/min

The HPLC analysis of Eliglustat tartrate premix of the present invention was analyzed by HPLC under the following conditions:

Apparatus: A liquid chromatographic system equipped with variable wavelength UV detector; Column: X-select CSH, C18 250*4.6 mm, 5 mm (or) equivalent; Column temperature: 45° C.; Wave length: 210 nm; Injection volume: 5 μL; Diluent: acetonitrile: water (80:20) % v/v; Buffer: transfer 2 ml of perchloric acid in 1000 mL of Milli-Q-water. Filter this solution through filter paper; Mobile phase-A: Buffer (100%) Mobile phase-B: Acetonitrile: Buffer (60:40)% v/v.
The process described in the present invention was demonstrated in examples illustrated below. These examples are provided as illustration only and therefore should not be construed as limitation of the scope of the invention:

### EXAMPLES

### Example-1: Preparation of Stable Eliglustat tartrate Premix

Eliglustat (50 gms) was dissolved in methanol (200 ml) at 25-30° C. and stirred for 10 min. L-(+)-Tartaric acid (10 gms) was added to the obtained solution at 25-30° C. and stirred for 30 min. Filtered the mixture and washed with methanol. Co-povidone (12.5 gms) was added to the above filtrate at 25-30° C. and stirred for 10 min at the same temperature. Magnesium aluminometa silicate & lactose monohydrate compact material (59.9 gms) was added to the above obtained mixture and stirred for 5 min. Methanol (100 ml) was added to the mixture and stirred for 10 min. Distilled off the solvent completely from the mixture under reduced pressure and then dried the obtained material to provide the title compound. (Yield: 118 gms, purity by HPLC: 99.86%)

PXRD pattern of the obtained stable Eliglustat tartrate premix according to the example-1 was illustrated in FIG. 1.
DSC thermogram of the obtained stable Eliglustat tartrate premix according to the example-1 was illustrated in FIG. 2.

### Example-2: Preparation of Stable Eliglustat tartrate Premix

Eliglustat (5 gms) was dissolved in methanol (20 ml) at 25-30° C. and stirred for 10 min. L-(+)-Tartaric acid (0.92 gms) was added to the obtained solution at 25-30° C. and stirred for 30 min. Filtered the mixture and washed with methanol. Co-povidone (1.25 gms) was added to the above filtrate at 25-30° C. and stirred for 10 min at the same temperature. Lactose monohydrate and magnesium aluminometa silicate (6.0 gms) was added to the above obtained mixture. Methanol (10 ml) was added to the obtained mixture and stirred for 10 min. Raised the temperature of the reaction mixture to 40-45° C. Distilled off the solvent completely from the mixture under reduced pressure and then dried the obtained material to provide the stable Eliglustat tartrate premix. (Yield: 13.0 gms, purity by HPLC: 99.97%)

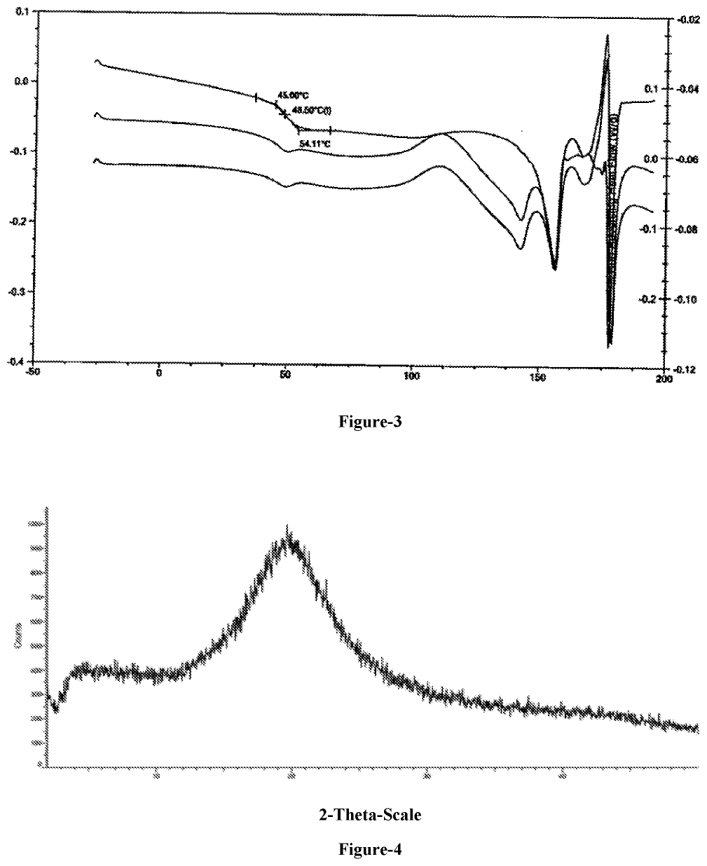

(Pyrrolidine amine impurity: Not detected; Hexanamide impurity: 0.01%; N-oxide impurity: 0.03%; Nonamide impurity: Not detected; Amide impurity: Not detected; Decanamide impurity: Not detected; Dioctanoyl impurity: Not detected; Eliglustat (S, S) isomer: Not detected)
The PXRD pattern of the obtained stable Eliglustat tartrate premix according to the example-2 was illustrated in FIG. 3.

### Example-3: Preparation of (E)-3-(2,3-dihydrobenzo[b][1,4]dioxin-6-yl)acrylic acid of formula (VII)

Malonic acid (63.3 gms) was added to 2,3-dihydrobenzo[b][1,4]dioxine-6-carbaldehyde (50 gms) in pyridine (300 ml) at 25-30° C. Heated the reaction mixture to 45-50° C. and stirred for 3 hours at same temperature. Malonic acid (63.3 gms) was added to the reaction mixture at 45-50° C. and stirred for 4 hours at same temperature. Malonic acid (31.6 gms) was added to the reaction mixture at 45-50° C. and stirred for 12-14 hours at same temperature. Cooled the obtained reaction mixture to 25-30° C. and added to chilled water (300 ml). Cooled the reaction mixture to 10° C. and stirred for 15 min at same temperature. Acidified the reaction mixture with HCl (150 ml) at 10-15° C. and stirred for 30 min at same temperature. Filtered the precipitated solid and washed with water. Methanol (250 ml) was added to the above obtained material and stirred for 2 hrs at 25-30° C. Filtered the precipitated solid and washed with methanol and then dried to get the title compound. (Yield: 50 gms)

### Example-4: Preparation of (E)-3-(2,3-dihydrobenzo[b][1,4]dioxin-6-yl)-1-(pyrrolidin-1-yl)prop-2-en-1-one of formula (VIII)

Thionyl chloride (30.2 ml) was added to (E)-3-(2,3-dihydrobenzo[b][1,4]dioxin-6-yl)acrylic acid (35 gms) in toluene (100 ml) at 25-30° C. under nitrogen atmosphere. Heated the reaction mixture to 60-65° C. and stirred for 2 hrs at same temperature. Pyrrolidine solution (36.1 gms dissolved in 20 ml of toluene) was added to the above reaction mixture at 15-20° C. and stirred for 15 min at same temperature. Heated reaction mixture to 25-30° C. and stirred for 2 hrs at same temperature. Water (300 ml) was added to the above reaction mixture and stirred for 10 min. Filtered the precipitated solid, washed with water and then dried to get the title compound. (Yield: 30.5 gm)

### Example-5: Preparation of 2-bromo-3-(2,3-dihydrobenzo[b][1,4]dioxin-6-yl)-3-hydroxy-1-(pyrrolidin-1-yl)propan-1-one of formula (IXa)

A mixture of (E)-3-(2,3-dihydrobenzo[b][1,4]dioxin-6-yl)-1-(pyrrolidin-1-yl)prop-2-en-1-one (30 gms), acetonitrile (318 ml), water (159 ml), NaIO₄ (7.4 gms), HCl (12.6 ml) and lithium bromide (12 gms) were stirred for 24 hrs at 25-30° C. Water (300 ml) and ethyl acetate (120 ml) were added to the above reaction mixture at 25-30° C. and stirred for 20 min at same temperature. Separated the organic and aqueous layers. Extracted the aqueous layer with ethyl acetate. Combined the total organic layers and washed with aqueous sodium bisulfate solution. Separated the organic and aqueous layers and washed the organic layer with 10% aqueous sodium chloride solution. Separated the organic layer and dried over sodium sulfate. Distilled off the solvent completely from the organic layer under reduced pressure. Cyclohexane (150 ml) was added to the above obtained material at 25-30° C. and stirred for 60 min at same temperature. Filtered the solid and washed with cyclohexane and then dried to get the title compound (Yield: 34.0 gms)

### Example-6: Preparation of 2-azido-3-(2,3-dihydrobenzo[b][1,4]dioxin-6-yl)-3-hydroxy-1-(pyrrolidin-1-yl)propan-1-one of formula (Xa)

Sodium azide (7.8 gms) was added to 2-bromo-3-(2,3-dihydrobenzo [b][1,4]dioxin-6-yl)-3-hydroxy-1-(pyrrolidin-1-yl)propan-1-one (33 gms) in dimethyl formamide (330 ml) at 25-30° C. Heated the reaction mixture to 60-65° C. and stirred for 3 hrs at the same temperature. Water (660 ml) and ethyl acetate (165 ml) were added to the reaction mixture and stirred for 20 min. Separated the both aqueous and organic layers. Extracted the aqueous layer with ethyl acetate. Combined the organic layers and washed with sodium chloride solution. Separated the both organic and aqueous layers and dried the organic layer with sodium sulfate. Distilled off the solvent completely from the organic layer under reduced pressure. Toluene (30 ml) and cyclohexane (20 ml) were added to the above obtained material and stirred for 4 hrs at 25-30° C. Filtered the solid, washed with mixture of toluene and cyclohexane and then dried to get the title compound (Yield: 10 gms)

### Example-7: Preparation of 2-amino-1-(2,3-dihydrobenzo[b][1,4]dioxin-6-yl)-3-(pyrrolidin-1-yl)propan-1-ol of formula (XIa)

To a mixture of tetrahydrofuran (50 ml) and LiAlH₄ solution (5.96 gms/ 60 ml, 10% in THF) added 2-azido-3-(2,3-dihydrobenzo[b][1,4]dioxin-6-yl)-3-hydroxy-1-(pyrrolidin-1-yl)propan-1-one (10 gms) in tetrahydrofuran (50 ml) at 25-30° C. and stirred for 3 hrs at same temperature. Cooled the reaction mixture to −25° C. to −30° C. Water (200 ml) was added to the reaction mixture at −25° C. to -30° C. and stirred for 20 min. Raised the temperature of the reaction mixture to 0-5° C. and acidified the reaction mixture with HCl (30 ml). Ethyl acetate (50 ml) was added to the reaction mixture and stirred for 20 min and settled the reaction mixture for 10 min and separated the organic and aqueous layers. Basified the aqueous layer with aqueous NaOH solution (15 gms dissolved in 30 ml of water). Ethyl acetate was added to the reaction mixture and stirred for 20 min. Separated the organic and aqueous layers. Extracted the aqueous layer with ethyl acetate and separated the organic and aqueous layers. Combined the both organic layers and washed with 10% aqueous sodium chloride solution. Separated the organic layer and dried over sodium sulfate and then distilled off the solvent completely under reduced pressure to get the title compound. (Yield: 6.0 gms)

### Example-8: Preparation of (1R,2R)-2-amino-1-(2,3-dihydrobenzo)[b][1,4]dioxin-6-yl)-3-(pyrrolidin-1-yl)propan-1-ol of formula (XIIb)

Toluene (50 ml) was added to 2-amino-1-(2,3-dihydrobenzo[b][1,4]dioxin-6-yl)-3-(pyrrolidin-1-yl)propan-1-ol (5.0 gms) and stirred for 10 min at 25-30° C. N-Acetyl leucine (3.1 gms) was added to the reaction mixture at 25-30° C. and stirred for 2 hrs at same tempera-ture. Ethyl acetate (50 ml) was added to the reaction mixture at 25-30° C. and stirred for 2 hrs at same temperature. Filtered the precipitated solid and washed with toluene and then dried to get the solid compound of salt of (1R,2R)-2-amino-1-(2,3-dihydrobenzo)[b][1,4]dioxin-6-yl)-3-(pyrrolidin-1-yl)propan-1-ol with N-acetyl leucine of formula (XIIa).

Water (10 ml) and CH₂Cl₂ (20 ml) were slowly added to the above obtained salt of formula (XIIa) and stirred for 5 min. 10% Aqueous NaOH solution was added to the reaction mixture and stirred for 20 min. Separated the organic and aqueous layers and washed the organic layer with water. Separated the organic layer and distilled off the solvent completely under reduced pressure from the organic layer to get the title compound. (Yield: 0.3 gms)

### Example-9: Preparation of pentafluorophenyl octanoate

Pentafluorophenol (50 gms) was added to dichloromethane (250 ml) at 25-30° C. under nitrogen atmosphere and stirred for 10 min at same temperature. Cooled the reaction mixture to 0-5° C. and added triethylamine (41.21 gms /55.69 ml) at same temperature. Octanoyl chloride (48.60 gms/53.23 ml) was slowly added to the above reaction mixture at 0-5° C. Heated the reaction mixture to 25-30° C. and stirred for 3 hrs at same temperature. Filtered the reaction mixture through hyflow bed and washed with dichloromethane. Basified the filtrate with aqueous sodium bicarbonate solution (5 gms dissolved in 100 ml of water) and stirred for 10 min. Separated the aqueous and organic layers and added water to the organic layer and stirred for 10 min. Separated the both aqueous and organic layers and dried the organic layer over sodium sulfate. Distilled off the solvent completely from the organic layer under reduced pressure to provide the title compound. (Yield: 76 gms)

### Example-10: Preparation of N-((1R,2R)-1-(2,3-dihydrobenzo[b][1,4]dioxin-6-yl)-1-hydroxy-3-(pyrrolidin-1-yl)propan-2-yl)octanamide of formula (I)

(1R,2R)-2-Amino-1-(2,3-dihydrobenzo [b][1,4]dioxin-6-yl)-3-(pyrrolidin-1-yl)propan-1-ol (0.6 gm) was added to dichloromethane (5 ml) and stirred for 10 min. Slowly heated the reaction mixture to 40-45° C. Pentafluorophenyl octanoate solution (0.7 gms in 5 ml of meth-ylene chloride) was added to the above reaction mixture and stirred 12 hrs. Basified the reac-tion mixture with 10% NaOH solution (0.5 gms dissolved in 5 ml of water) and stirred for 20 min. Separated the organic and aqueous layers. Water (5 ml) was added to the organic layer and stirred for 20 min. Separated the organic and aqueous layers. Distilled off the solvent completely from the organic layer under reduced pressure. Cooled the obtained compound to 25-30° C. Methyl tert-butyl ether (5 ml) was added to the reaction mixture and then cooled to 0-5° C. Acidified the reaction mixture with 6M HCl. Heated the reaction mixture to 25-30° C. and stirred for 20 min at same temperature. Separated the organic and aqueous layers. Cooled the aqueous layer to 0-5° C. and basified with 10% aqueous sodium hydroxide solution (0.5 gms dissolved in 5 ml of water). Methylene chloride (15 ml) was added to the reaction mixture at 25-30° C. and stirred for 20 min at same temperature. Separated the organic and aqueous layers. Distilled off the solvent completely from the organic layer under reduced pressure. Ethyl acetate (1 ml) and n-heptane (17 ml) were added to the obtained compound and heated reaction mixture to 90-95° C. and stirred for 40 min at same temperature. Cooled the reaction mixture to 60-65° C. and decanted into another round bottom flask. Cooled the reaction mixture to 25-30° C. and stirred for 40 min at same temperature. Filtered the precipitated solid, washed with n-heptane and then dried to get the title compound (Yield: 0.25 gms)

### Example-11: Preparation of (R)-tert-butyl (3-hydroxy-1-(methoxy(methyl)amino)-1-oxopropan-2-yl)carbamate

(R)-2-((tert-Butoxycarbonyl)amino)-3-hydroxypropanoic acid (100 gms) was added to dichloromethane (1000 ml) under nitrogen atmosphere and stirred for 10-15 min at 25-30° C. Cooled the reaction mixture to −15 to −10° C. N,O-Dimethylhydroxylamine hydrochloride (45.4 gms), N-methylmorpholine (123.2 gms) and 1-ethyl-3-(3-dimethylaminopropyl) carbodiimide hydrochloride (95.2 gms) were added and stirred the reaction mixture for 2 hr at −15 to −10° C. Acidified the reaction mixture with 1N hydrochloric acid solution (600 ml) and stirred for 15 min. Separated the both aqueous and organic layers. Basify the organic layer with 5% aqueous sodium bicarbonate solution and stirred for 15 min. Separated the organic and aqueous layers and distilled off the solvent completely from the organic layer under reduced pressure. Petroleum ether (100 ml) was added to the above obtained material at 25-30° C. and stirred the reaction mixture for 60 min at same temperature. Filtered the precipitated solid and dried to get the title compound. (Yield: 92.0 gms)

### Example-12: Preparation of tert-butyl 4-(methoxy(methyl)carbamoyl)-2,2-dimethyloxazolidine-3-carboxylate

(R)-Tert-butyl (3-hydroxy-1-(methoxy(methyl)amino)-1-oxopropan-2-yl)carbamate (150 gms) and acetone (1995 ml) were charged into a round bottom flask. 2,2-Dimethoxypropane (530 gms/627 ml) was added to the mixture and stirred for 15 min. Boron trifluoride etherate (5.65 gms) was added to the reaction mixture at 25-30° C. and stirred for 3 hrs. Triethylamine (2.01 ml) was added to the above reaction mixture and stirred for 15 min. Distilled off the solvent completely from reaction mixture under reduced pressure to get the title compound. (Yield: 170.0 gms).

### Example-13: Preparation of (R)-tert-butyl 4-formyl-2,2-dimethyloxazolidine-3-carboxylate

Tert-butyl 4-(methoxy(methyl)carbamoyl)-2,2-dimethyloxazolidine-3-carboxylate (170 gms) was added to toluene (870 ml) at 25-30° C. and stirred for 15 min. Cooled the reaction mixture to 0-5° C. Sodium bis(2-methoxyethoxy)aluminum hydride (348.7 ml) was slowly added to the reaction mixture at 0-5° C. and stirred for 3 hrs at the same temperature. The obtained reaction mixture was slowly added to a pre-cooled aqueous sodium potassium tartrate solution (100 gms in 1044 ml of water). Heated the reaction mixture to 25-30° C. and stirred for 45 min. Filtered the reaction mixture through hyflow bed and washed the bed with toluene. Separated the both organic and aqueous layers. Distilled off the solvent completely from the organic layer under reduced pressure to get the title compound. (Yield: 100.0 gms).

### Example-14: Preparation of (R)-tert-butyl 4-((R)-(2,3-dihydrobenzo[b][1,4]dioxin-6-yl)(hydroxy)methyl)-2,2-dimethyloxazolidine-3-carboxylate

Tetrahydrofuran (75 ml) was added to 6-bromo-2,3-dihydrobenzo[b][1,4]dioxine (15.4 gms) at 25-30° C. Cooled the reaction mixture to −75° C. and added n-BuLi (81.7 ml) and stirred for 30 min at the same temperature. To this reaction mixture slowly added a solution of tert-butyl 4-formyl-2,2-dimethyloxazolidine-3-carboxylate (15 gms in 75 ml of THF) at −75° C. and stirred for 20 min at the same temperature. Heated the reaction mixture to 25-30° C. and stirred for 10 hrs. 5% Aqueous ammonium chloride solution (3.5 gms in 75 ml of water) was added to the reaction mixture at 0-5° C. and stirred for 20 min at the same temperature. Heated the reaction mixture to 25-30° C. Ethyl acetate (150 ml) was added to the reaction mixture and stirred for 15 min. Separated the both aqueous and organic layers and extracted the aqueous layer with ethyl acetate. Combined the total organic layers and distilled off the solvent completely from the organic layer under reduced pressure to provide the title compound. (Yield: 17.0 gms).

### Example-15: Preparation of tert-butyl ((1R,2R)-1-(2,3-dihydrobenzo[b][1,4]dioxin-6-yl)-1,3-dihydroxypropan-2-yl)carbamate

(R)-Tert-butyl 4-((R)-(2,3-dihydrobenzo[b][1,4]dioxin-6-yl)(hydroxy)methyl)-2,2-dimethyloxazolidine-3-carboxylate (8.0 gms) and methanol (40 ml) were stirred for 15 min at 25-30° C. Cooled reaction mixture to 0-5° C. p-Toluenesulfonic acid (2.1 gms) was added to the mixture and stirred for 6 hrs at the same temperature. Slowly added 5% aqueous ammonium chloride solution (8 gms in 80 ml of water) and dichloromethane (40 ml). Heated the reaction mixture to 25-30° C. and stirred for 15 min. Both the organic layer and aqueous layer were separated from the filtrate. Extracted the aqueous layer with dichloromethane. Combined the total organic layers and distilled off the solvent completely from the organic layer under reduced pressure to provide the title compound. (Yield: 5.0 gms).

### Example-16: Preparation of (2R,3R)-2-((tert-butoxycarbonyl)amino)-3-(2,3-dihydrobenzo [b][1,4]dioxin-6-yl)-3-hydroxypropyl methanesulfonate

Triethylamine (4.6 gms) was added to tert-butyl ((1R,2R)-1-(2,3-dihydrobenzo[b][1,4]dioxin-6-yl)-1,3-dihydroxypropan-2-yl)carbamate (5.0 gms) in dichloromethane (25 ml) at 0-5° C. and stirred for 15 min at same temperature. Methane sulfonyl chloride (2.1 gms) was added to the reaction mixture at 0-5° C. and stirred for 60 min at the same temperature. Water (50 ml) was added to the above reaction mixture and heated to 25-30° C. and stirred for 15 min. Separated the aqueous and organic layers and extracted the aqueous layer with dichloromethane. Combined the total organic layers and washed with water. Distilled off the solvent completely from the organic layer under reduced pressure to get the title compound. (Yield: 4.2 gms).

### Example-17: Preparation of tert-butyl ((1R,2R)-1-(2,3-dihydrobenzo[b][1,4]dioxin-6-yl)-1-hydroxy-3-(pyrrolidin-1-yl)propan-2-yl)carbamate

(2R,3R)-2-((Tert-butoxycarbonyl)amino)-3-(2,3-dihydrobenzo [b][1,4]dioxin-6-yl)-3-hydroxypropyl methanesulfonate (4.0 gms) was added to pyrrolidine (10 ml) at 25-30° C. Heated the reaction mixture to 45° C. and stirred for 6 hrs. Cooled the reaction mixture to 25-30° C. Water (40 ml) and dichloromethane (40 ml) were added to the reaction mixture and stirred for 15 min. Separated the both organic and aqueous layers. Extracted the aqueous layer with dichloromethane. Combined the total the organic layers and washed with water. Distilled off the solvent completely from the organic layer under reduced pressure to provide the title compound. (Yield: 2.8 gms).

### Example-18: Preparation of (1R,2R)-2-amino-1-(2,3-dihydrobenzo[b][1,4]dioxin-6-yl)-3-(pyrrolidin-1-yl)propan-1-ol of formula (XIIb)

Tert-butyl ((1R,2R)-1-(2,3-dihydrobenzo[b][1,4]dioxin-6-yl)-1-hydroxy-3-(pyrrolidin-1-yl)propan-2-yl)carbamate (2.5 gms) was added to dichloromethane (25 ml) at 25-30° C. Hydrochloric acid (10 ml) was added to the mixture and stirred for 45 min. Heated the mixture to 45-50° C. and stirred for 6 hrs. Cooled the reaction mixture to 30° C. and separated the both aqueous and organic layers. The aqueous layer was extracted with dichloromethane. Basified the aqueous layer with aqueous sodium hydroxide solution and added dichloromethane (10 ml) and stirred for 15 min. Separated the organic and aqueous layers. Combined the total organic layers and distilled off the solvent completely from the organic layer under reduced pressure to get the title compound. (Yield: 0.8 gms).

### Example-19: Preparation of Eliglustat tartrate of Formula (Ia)

Eliglustat (5.0 gms) was added to ethyl acetate (50 ml) at 25-30° C. and stirred for 10 min. L(+)-Tartaric acid solution (0.93 gms dissolved in 50 ml of acetone) was slowly added to the above mixture and stirred for 60 min. Filtered the precipitated solid, washed with ethyl acetate and then dried to get title compound. (Yield: 5.2 gms)

### Example-20: Preparation of Eliglustat tartrate of Formula (Ia)

Eliglustat (1 gm) was added to acetone (5 ml) at 25-30° C. (2R,3R)-2,3-dihydroxysuccinic acid in acetone (0.185 gms in 5 ml of acetone) was slowly added to the above reaction mixture at 25-30° C. and stirred for 2 hrs at same temperature. Filtered the precipitated solid, washed with acetone and then dried to provide the title compound (Yield: 0.8 gms).

### Example-21: Preparation of solid dispersion of Eliglustat hemi (2R,3R)-2,3-dihydroxysuccinic acid with HPMC-AS (1:1)

Eliglustat (5 gms) and hydroxy propyl methyl cellulose- acetyl succinate (HPMC-AS) (5 gms) were dissolved in methanol (100 ml) at 25-30° C. and stirred for 15 min. Filtered the mixture for particle free solution. Distilled off the solvent completely from the mixture under reduced pressure and then dried to get the title compound (Yield: 8 gms)

The PXRD pattern of the above obtained compound was illustrated in FIG. 4.

### Example-22: Preparation of solid dispersion of Eliglustat hemi (2R,3R)-2,3-dihydroxysuccinic acid with HPMC-E5 (7.5:2.5)

Eliglustat (5 gms) was added to methanol (50 ml) at 25-30° C. and stirred for 10 min at same temperature. HPMC-E5 solution (1.6 gms dissolved in mixture of methanol (16 ml) and dichloromethane (16)) was added to the above solution at 25-30° C. and stirred for 10 min. Filtered the mixture for particle free solution. Distilled off the solvent completely from the mixture under reduced pressure and then dried to get the title compound (Yield: 5 gms)

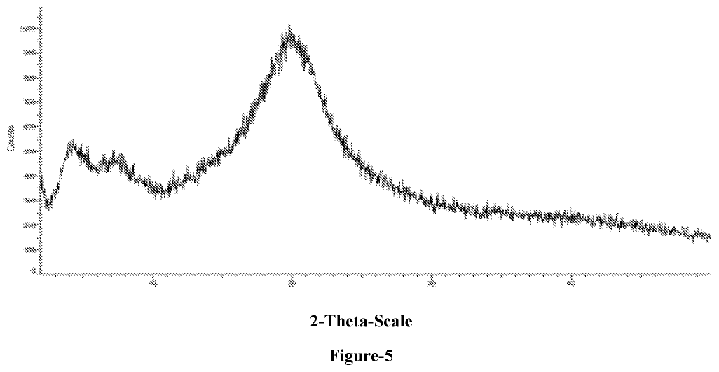

The PXRD pattern of the above obtained compound was illustrated in FIG. 5.

1. Stable Eliglustat tartrate premix, comprising: at least two or more pharmaceutical acceptable excipients.

2. Stable Eliglustat tartrate premix of claim 1, prepared by combining Eliglustat, tartaric acid and at least two or more pharmaceutical acceptable excipients.

3. According to claim 1, wherein the pharmaceutical acceptable excipients are selected from binders, diluents, disintegrants, surfactants and lubricants.

4. According to claim 3, binders are selected from polyvinylpyrolidone, copovidone, cyclodextrin, hydroxypropylmethyl cellulose; diluents selected from anhydrous lactose, lactose monohydrate, modified lactose; disintegrants selected from magnesium aluminometa silicate (or magnesium aluminum silicate); lubricants selected from magnesium stearate, stearic acid, palmitic acid, talc and aerosil; surfactants selected from polysorbate 80, polyoxyethylene sorbitan, polyoxyethylene-polyoxy-propylene copolymer and sodium lauryl sulphate.

5. Stable Eliglustat tartrate premix, characterized by any one from the following:
a) X-Ray Diffraction Pattern as illustrated by FIG. 1.
b) Differential Scanning calorimetry which exhibits glass transition between about 45° C. to about 55° C.
c) having the bulk density between about 0.1 gm/ml to about 0.9 gm/ml.

6. Stable Eliglustat tartrate premix of claim 5 having bulk density between about 0.5 gm/ml to about 0.7 gm/ml.

7. Stable Eliglustat tartrate premix of claim 5 comprise of co-povidone, lactose monohydrate and magnesium aluminometa silicate.

8. Stable Eliglustat tartrate premix of claim 5 contains about 26.5% of lactose monohydrate, about 19.0% of magnesium aluminometa silicate and about 9.5% of copovidone.

9. Stable Eliglustat tartrate premix contains less than about 0.15% of one or more impurities selected from pyrrolidine amine impurity, hexanamide impurity, N-oxide impurity, nonamide impurity, amide impurity, decanamide impurity and dioctanoyl impurity.

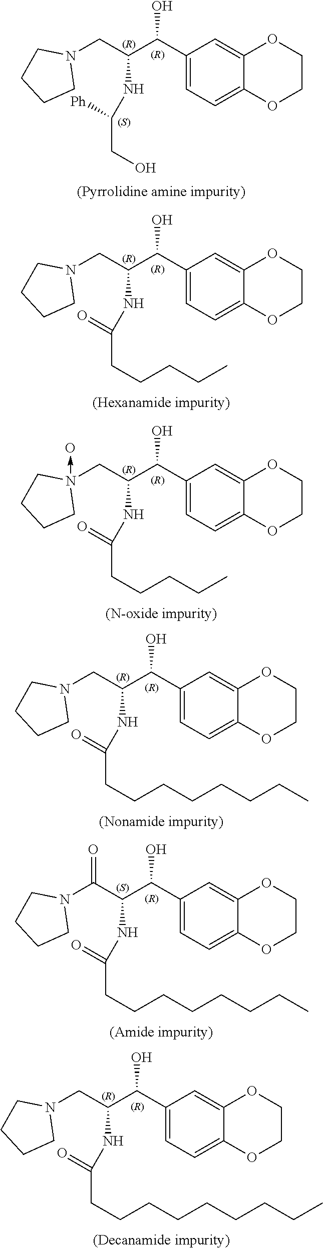

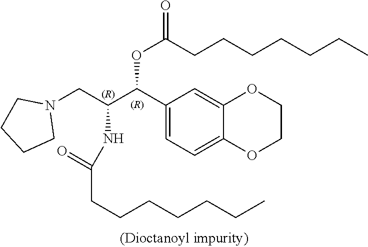

10. Stable Eliglustat tartrate premix of claim 9 substantially free from one or more impurities selected from pyrrolidine amine impurity, nonamide impurity, amide impurity, decanamide impurity and dioctanoyl impurity.

11. A process for the preparation of stable Eliglustat tartrate premix of claim 5, comprising of:
a) dissolving Eliglustat free base in a suitable solvent,
b) adding tartaric acid to the solution obtained in step-a),
c) adding two or more pharmaceutical acceptable excipients to the solution obtained in step-b),
d) isolating stable Eliglustat tartrate premix.

12. The process according to claim 11, wherein, in step-a) the suitable solvent is selected from alcoholic solvents, ester solvents, ether solvents, hydrocarbon solvents, chloro solvents, polar aprotic solvents; nitrile solvents, ketone solvents, polar solvents or mixtures thereof.

13. The process according to claim 11, wherein in step-c) pharmaceutically acceptable excipients selected from binders, diluents, disintegrants, surfactants and lubricants.

14. The process according to claim 11, wherein, in step-d) isolation carried out by removal of the solvent by the known methods like distillation, decantation, filtration and then drying or any other methods known in the art.

15. A process for the preparation of stable Eliglustat tartrate premix, comprising:
a) dissolving Eliglustat free base in methanol,
b) adding tartaric acid to a solution obtained in step-a)
c) adding co-povidone to the solution obtained in step-b),
d) adding magnesium aluminometa silicate & lactose monohydrate to the mixture obtained in step-c),
e) removing the solvent from the mixture obtained step-c) to provide stable Eliglustat tartrate premix.

16-48. (canceled)
